(19) United States
(12) Patent Application Publication
(10) Pub. No.: US 2006/0107955 A1
(43) Pub. Date: May 25, 2006

(54) Home oxygen-compression apparatus

(76) Inventor: Chen-Bong Cheng, Kowloon (HK)

Correspondence Address: HERSHKOVITZ & ASSOCIATES, 1725 I STREET NW, SUITE 300, WASHINGTON, DC, 20006, US

(21) Appl. No.: 10/992,764

(22) Filed: Nov. 22, 2004

(51) Int. Cl. A61M 16/00; A62B 7/10

(52) U.S. Cl. 128/205.12; 128/204.22

(57) ABSTRACT

An oxygen-compression apparatus has a driving device, an output shaft, two crank devices, two cylinders and multiple hoses. The crank devices are attached respectively to two ends of the output shaft, and each crank device has a rotating arm and a pushing arm pivotally attached to the rotating arm. The cylinders are connected respectively to the crank devices. Each cylinder has a housing and a piston rod. The piston rod has a first end extending into the housing to form a compression chamber in the housing and a second end pivotally connected to one of the pushing arms. The hoses are connected between the compression chambers of the cylinders. Accordingly, the oxygen-compression apparatus can conveniently pump oxygen into a gas tank at a desired pressure and can simultaneously apply oxygen to a user with an auxiliary output hose.

### BACKGROUND OF THE INVENTION

1. Field of the Invention

The present invention relates to an oxygen-compression apparatus, and more particularly to a domestic oxygen-compression apparatus that can conveniently store oxygen in a tank with a desired pressure.

2. Description of Related Art

Oxygen is commonly applied to assist a patient's breathing, and home oxygen concentrators have been utilized to supply patients with oxygen at home. A conventional apparatus for producing oxygen is disclosed in U.S. Pat. No. 5,988,165, entitled “Apparatus and Method for Forming Oxygen-Enriched Gas and Compression Thereof for High-Pressure Mobile Storage Utilization”. The '165 Patent can produce enriched oxygen and compresses the oxygen to portable container under a desired high pressure, i.e. above 2000 psi, such that the patient can carry the container with compressed oxygen to any desired location for use. The '165 patent provides a compressor composed of a crankshaft, multiple connecting rods, multiple cylinders and multiple pistons. The connecting rods are connected to the crankshaft. The pistons are connected respectively to the connecting rods and extend respectively into the cylinders. With the rotation of the crankshaft, the pistons will compress oxygen in the cylinder with the transmission of the connecting rods, such that the oxygen will be compressed to a desired high pressure and is then collected in a gas tank. However, the compressor of the '165 patent takes up a large space and is inconvenient in operation.

Another apparatus is disclosed in U.S. Pat. No. 5,345,361, entitled “Energy Recovering Pressure Balance Scheme for a Combination Pressure Swing Absorber with a Boost Compressor”. The '361 patent discloses a pressure booster composed of two stage compression cylinders and a driving cylinder with a driving shaft. The driving shaft has two ends extending respectively into the stage compression cylinders. The driving cylinder actuates the driving shaft to move forward and backward so as to compress the oxygen in the stage compression cylinder alternately, such that the oxygen is compressed to a desired high pressure and collected in a gas tank.

However, the '361 patent needs multiple switches to control the flow of compressed air applied to the driving cylinder for moving the driving shaft, its booster structure and the cost of manufacturing the booster is high. In addition, to compress oxygen to a desired high pressure, i.e. above 2000 psi with the booster of the '361 patent takes a long time, and the operation of the '361 patent is inefficient.

To overcome the shortcomings, the present invention tends to provide an oxygen-compression apparatus to mitigate or obviate the aforementioned problems.

### SUMMARY OF THE INVENTION

The main objective of the invention is to provide an oxygen-compression apparatus that can conveniently pump oxygen into a gas tank at a desired pressure and can simultaneously apply oxygen to a user with an auxiliary output hose. The oxygen-compression apparatus has a driving device, an output shaft, two crank devices, two cylinders, a connecting hose, an input hose and an output hose. The driving device has a driving shaft. The output shaft is connected to the driving shaft through a speed-reduction device and has two ends. The crank devices are attached respectively to the ends of the output shaft, and each crank device has a rotating arm attached to one end of the output shaft and a pushing arm pivotally attached to the rotating arm. The cylinders are connected respectively to the crank devices. Each cylinder has a housing with an inner space and a piston rod. The piston rod has a first end extending into the inner space of the housing to form a compression chamber in the inner space and a second end pivotally connected to the pushing arm of one of the crank devices. The connecting hose is connected between the compression chambers of the cylinders. The input hose is connected to the compression chamber of one of the cylinders and is adapted to be connected to an oxygen source to make the cylinder serve as a first stage cylinder. The output hose is connected to the compression chamber of the other cylinder whereby the cylinder serves as a second stage cylinder.

Other objects, advantages and novel features of the invention will become more apparent from the following detailed description when taken in conjunction with the accompanying drawings.

### BRIEF DESCRIPTION OF THE DRAWINGS

FIG. 1 is a schematic view of an oxygen-compression apparatus in accordance with the present invention;

FIG. 2 is a perspective view of the oxygen-compression apparatus in FIG. 1;

FIG. 3 is a top plan view of the oxygen-compression apparatus in FIG. 2;

FIG. 4 is a side plan view of the oxygen-compression apparatus in FIG. 2;

FIG. 5 is an operational side plan view of the oxygen-compression apparatus in FIG. 4 showing that the compression chamber of the cylinder is enlarged with the transmission of the corresponding crank device; and

FIG. 6 is an operational side plan view of the oxygen-compression apparatus in FIG. 4 showing that the compression chamber of the cylinder is reduced with the transmission of the corresponding crank device.

### DETAILED DESCRIPTION OF PREFERRED EMBODIMENT

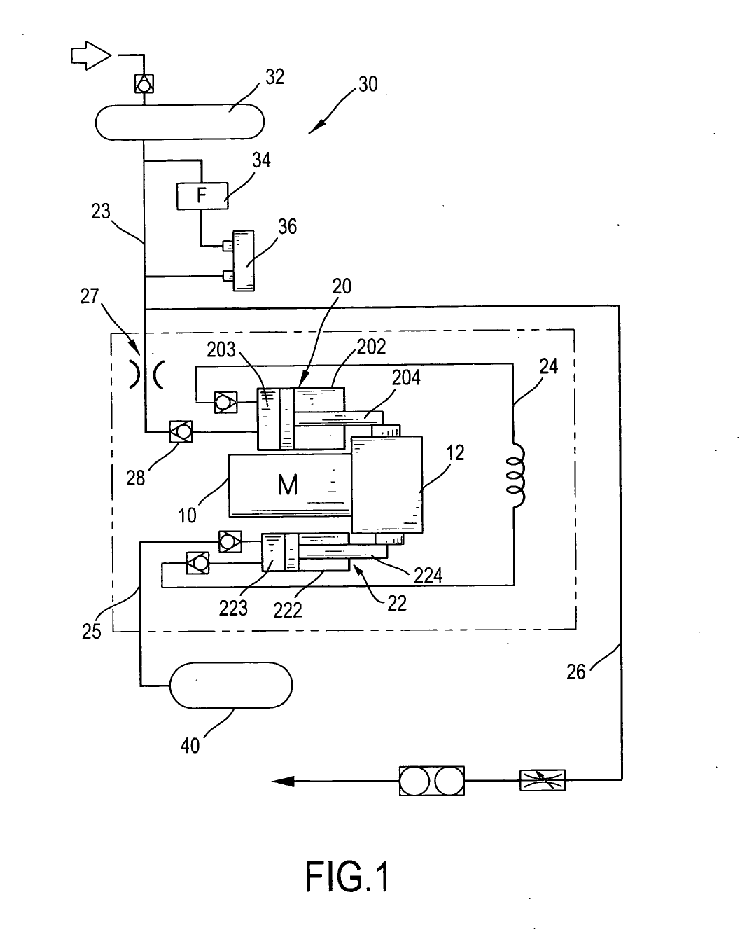

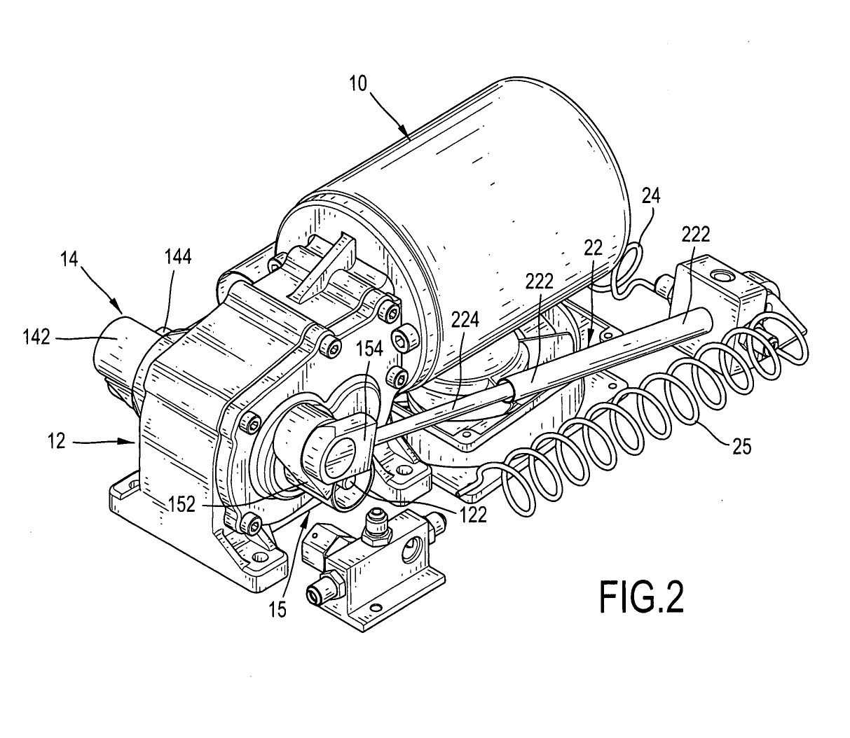

With reference to FIGS. 1 to 3, an oxygen-compression apparatus in accordance with the present invention comprises a driving device (10), an output shaft (122), two crank devices (14,15), two cylinders (20,22) and multiple hoses (23,24,25).

The driving device (10) has a driving shaft (11) and may be a motor. The output shaft (12) is connected to the driving shaft (11) through a speed-reduction device (12) and has two ends. In an optional embodiment, the speed-reduction device (12) is a gear box comprising multiple gears engaging with each other. With the arrangement of the speed-reduction device (12), the speed of the output shaft (122) is reduced to a desired low speed.

The crank devices (14,15) are attached respectively to the ends of the output shaft (122) and each has a rotating arm (142,152) and a pushing arm (144,154). The rotating arm (142,152) is attached to one end of the output shaft (122), and the pushing arm (144,154) pivotally attached to the rotating arm (142,152). In a preferred embodiment, the rotating arms (142,152) of the crank devices (14,15) respectively extend toward opposite directions.

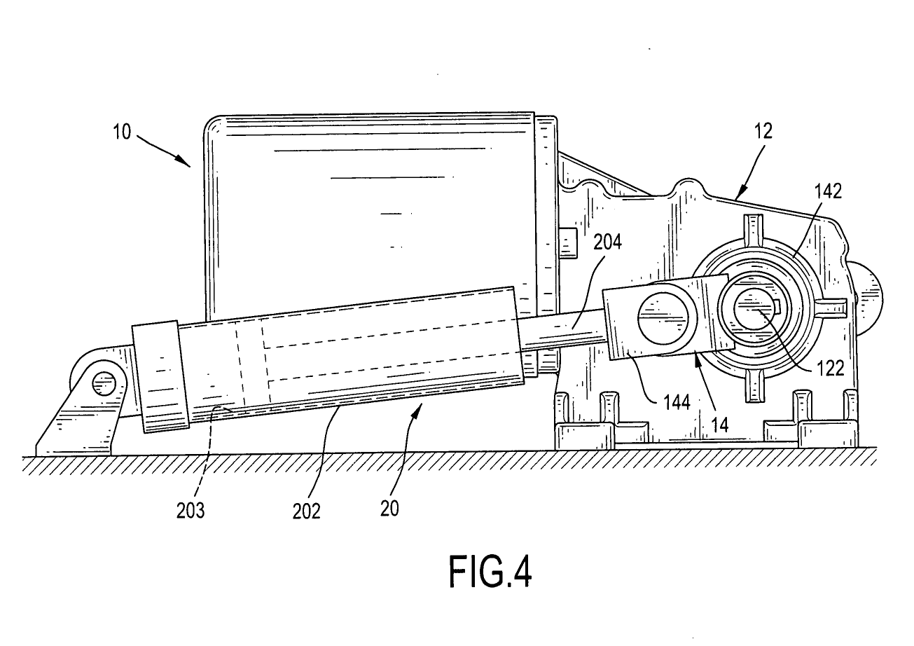

With further reference to FIG. 4, the cylinders (20,22) are connected respectively to the crank devices (14,15). Each cylinder (20,22) has a housing (202,222) and a piston rod (204,224). The housing (202,222) has an inner space, and the piston rod (204,224) has a first end extending into the inner space of the housing (202,222) to form a compression chamber (203,223) in the housing (202,222). The piston rod (204,224) further has a second end pivotally connected to the pushing arm (144,154) of one of the crank devices (14,15).

In addition, the apparatus further has a base to support the driving device (10), the speed-reduction device (12) and the cylinders (20,22). The housings (202,222) of the cylinders (202,222) are pivotally attached to the base.

The hoses comprise a connecting hose (24), an input hose (23) and an output hose (25). The connecting hose (24) is connected between the compression chambers (203,223) of the cylinders (20,22). The input hose (23) is connected to the compression chamber (203) of one of the cylinders (20) and is adapted to be connected to an oxygen source to make the cylinder (20) serve as a first stage cylinder (20a). The output hose (25) is connected to the compression chamber (223) of the other cylinder (22) and is adapted to be connected to a gas tank (40) to make the cylinder (22) serve as a second stage cylinder (22a). In a preferred embodiment, the inner space of the housing (202) of the first stage cylinder (20) has an inner diameter larger than that of the inner space of the housing (222) of the second stage cylinder (22). The first stage cylinder (20) has a compression ratio of 15:1, and the second stage cylinder (22) has a compression ratio of 10:1.

In addition, as shown in FIG. 1, multiple check valves (28) are attached respectively to the hoses (23,24,25) to form a one-way passage between the hoses (23,24,25) and the compression chambers (203,223) of the cylinders (20,22).

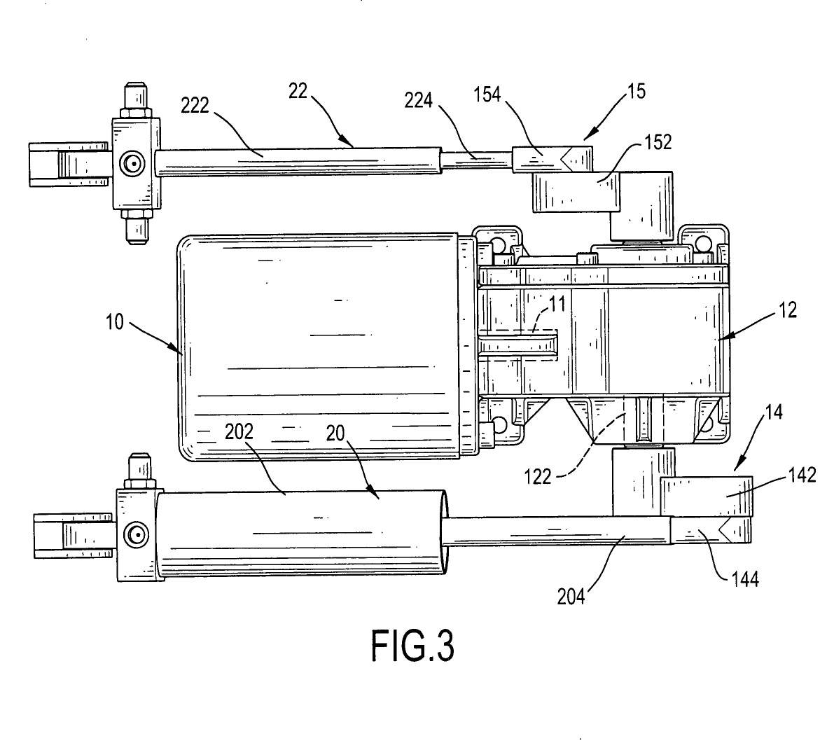

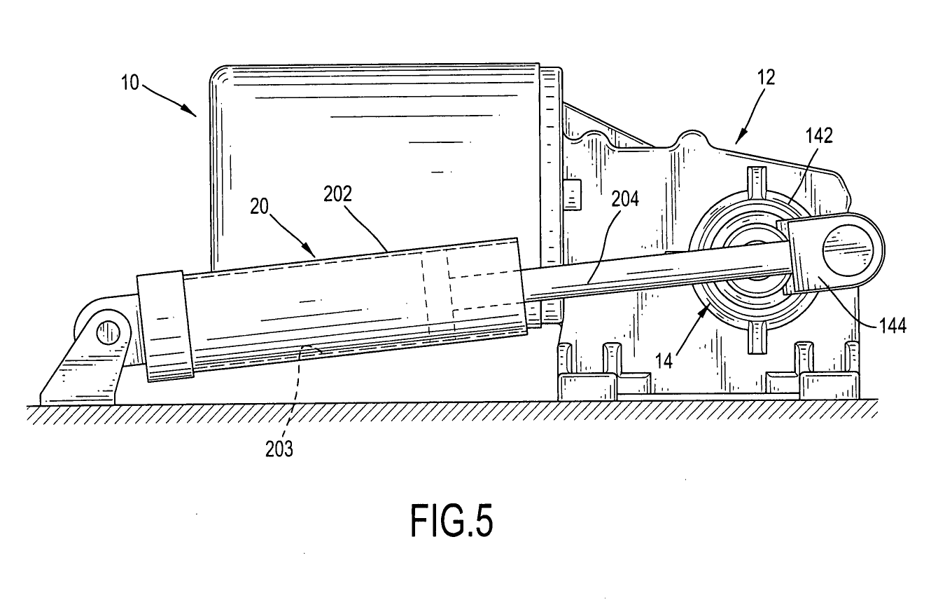

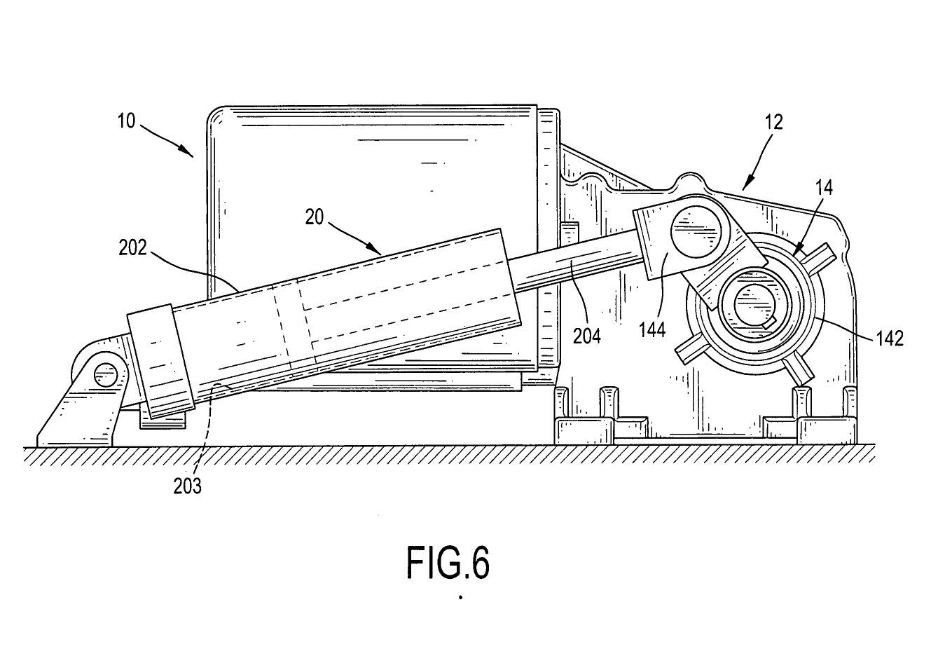

In such an arrangement, when the driving device (10) is switched on, the output shaft (122) rotates at a low speed and high torque and the rotating arms (142,152) of the crank devices (14,15) rotate with the output shaft (122). The pushing arms (144,154) will rotate with rotating arms (142,152) and push or pull the connecting pistons (204,224). When the output shaft (122) rotates to a position where the pushing arm (144) pulls the piston (204) away from the housing (202) of the first stage cylinder (20), with reference to FIG. 5, the compression chamber (203) is enlarged and the pressure in the compression chamber (203) is reduced. The oxygen provided from the oxygen source will be routed and sucked into the compression chamber (203) in the first stage cylinder (20) through the input hose (23). With the rotation of the output shaft (122), with reference to FIG. 6, the piston (204) of the first stage cylinder (20) will be pushed by the corresponding pushing arm (144) and the space of the compression chamber (203) is reduced. Thus, the pressure in the compression chamber (203) in the first stage cylinder (20) increases, and the pressurized oxygen exits the first stage cylinder (20) and enters the compression chamber (223) in the second stage cylinder (22) through the connecting hose (24). At this time, with reference to FIGS. 1 and 3, the piston (224) on the second stage cylinder (22) is pulled by the corresponding pushing arm (154) because of the opposition extension of the rotating arms (142,152) of the crank devices (14,15). Consequently, the compression chamber (223) in the second stage cylinder (22) is enlarged and provides an extraction effect to the pressurized oxygen out of the first stage cylinder (20). When the output shaft (122) rotates to a position where the pushing arm (154) pushes the piston (224) into the housing (222) of the second stage cylinder (22), the oxygen in the pressing chamber (223) will be compressed and exits the cylinder (22) under high pressure. Accordingly, the compressed oxygen can be routed to and collected into a gas tank (40) with a desired high pressure through the output hose (25), such that the user can conveniently store or carry the compressed oxygen to any desired location.

With such an apparatus, an oxygen-compression apparatus with a simple structure is provided, and the cost for manufacturing the apparatus is low. In addition, because the cylinders (20,22) can be alternately compressed with the crank devices (14,15), to compress the oxygen to a desired high pressure is efficient and takes a short time. Therefore, the user can easily store pressurized oxygen in gas tanks at home.

Furthermore, with reference to FIG. 1, an auxiliary output hose (26) is attached to the input hose (23), such that oxygen supplied from the oxygen source can be applied to a user directly at a low pressure. The user can breathe and store oxygen with the apparatus at the same time, such that the use of the oxygen-compression apparatus in accordance with the present invention is versatile.

In addition, an oxygen concentration detecting device (30) is attached to the input hose (23) to detect the concentration of the oxygen supplied from the oxygen source. The detecting device (30) comprises a buffer tank (32), a filter (34), an oxygen sensing unit (36) and a restrictor (27). The buffer tank (32) is connected to the input hose (32). The filter (34) is connected to the buffer tank (32). The oxygen sensing unit (36) is connected to the filter (34) to detect the concentration of the oxygen applied into the input hose (23). The restrictor (27) is connected to the input hose (23), is electrically connected to the oxygen sensing unit (36) and can be a switch to close the passage of the input hose (23) until the restrictor (27) is opened.

With such an oxygen concentration detecting device (30), oxygen will be not applied to the first stage cylinder (20) before the concentration of oxygen reaches a desired level. When the concentration of oxygen supplied from the oxygen source and detected by the oxygen sensing unit (36) reaches a predetermined level, the oxygen sensing unit (36) sends a signal to open the restrictor (37). Thereafter, oxygen at a desired concentration is applied to the cylinders (20,22) and is compressed to a desired pressure with the apparatus.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. An oxygen-compression apparatus comprising:
a driving device with a driving shaft;
an output shaft connected to the driving shaft through a speed-reduction device and having two ends;
two crank devices attached respectively to the ends of the output shaft and each having a rotating arm attached to one end of the output shaft and a pushing arm pivotally attached to the rotating arm;
two cylinders connected respectively to the crank devices and each having
a housing with an inner space; and
a piston rod having a first end extending into the inner space of the housing to form a compression chamber in the inner space and a second end pivotally connected to the pushing arm of one of the crank devices;
a connecting hose connected between the compression chambers of the cylinders;
an input hose connected to the compression chamber of a first of the cylinders and adapted to be connected to an oxygen source whereby the first cylinder serves as a first stage cylinder;
an output hose connected to the compression chamber of the second cylinder whereby the second cylinder serves as a second stage cylinder.

2. The apparatus as claimed in claim 1, wherein the speed-reduction device is a gear box.

3. The apparatus as claimed in claim 2, wherein the rotating arms of the crank devices respectively extend toward opposite directions.

4. The apparatus as claimed in claim 3 further comprising a base to support the driving device and the cylinders,
wherein the housings of the cylinders are pivotally attached to the base.

5. The apparatus as claimed in claim 4, wherein the inner space of the housing of the first stage cylinder has an inner diameter larger than that of the inner space of the housing of the second stage cylinder.

6. The apparatus as claimed in claim 5, wherein the first stage cylinder has a compression ratio of 15:1; and
the second stage cylinder has a compression ratio of 10:1.

7. The apparatus as claimed in claim 6 further comprising multiple check valves attached respectively to the hoses to form a one-way passage between the hoses and the compression chambers of the cylinders.

8. The apparatus as claimed in claim 7 further comprising an auxiliary output hose attached to the input hose to apply oxygen to a user directly.

9. The apparatus as claimed in claim 8 further comprising
an oxygen concentration detecting device attached to the input hose to detect the concentration of the oxygen supplied from the oxygen source, and the detecting device comprising
a buffer tank connected to the input hose;
a filter connected to the buffer tank;
an oxygen sensing unit connected to the filter to detect the concentration of the oxygen in the input hose; and
a restrictor connected to the input hose and electrically connected to the oxygen sensing unit.

10. The apparatus as claimed in claim 1, wherein the rotating arms of the crank devices respectively extend toward opposite directions.

11. The apparatus as claimed in claim 1 further comprising a base to support the driving device and the cylinders,
wherein the housings of the cylinders are pivotally attached to the base.

12. The apparatus as claimed in claim 1, wherein the inner space of the housing of the first stage cylinder has an inner diameter larger than that of the inner space of the housing of the second stage cylinder.

13. The apparatus as claimed in claim 12, wherein the first stage cylinder has a compression ratio of 15:1; and
the second stage cylinder has a compression ratio of 10:1.

14. The apparatus as claimed in claim 1 further comprising multiple check valves attached respectively to the hoses to form a one-way passage between the hoses and the compression chambers of the cylinders.

15. The apparatus as claimed in claim 14 further comprising an auxiliary output hose attached to the input hose to apply oxygen to a user directly.

16. The apparatus as claimed in claim 15 further comprising
an oxygen concentration detecting device attached to the input hose to detect the concentration of the oxygen supplied from the oxygen source, and the detecting device comprising
a buffer tank connected to the input hose;
a filter connected to the buffer tank;
an oxygen sensing unit connected to the filter to detect the concentration of the oxygen in the input hose; and
a restrictor connected to the input hose and electrically connected to the oxygen sensing unit.

17. The apparatus as claimed in claim 1 further comprising an auxiliary output hose attached to the input hose to apply oxygen to a user directly.

18. The apparatus as claimed in claim 1 further comprising
an oxygen concentration detecting device attached to the input hose to detect the concentration of the oxygen supplied from the oxygen source, and the detecting device comprising
a buffer tank connected to the input hose;
a filter connected to the buffer tank;
an oxygen sensing unit connected to the filter to detect the concentration of the oxygen in the input hose; and
a restrictor connected to the input hose and electrically connected to the oxygen sensing unit.
